(19) United States
(12) Patent Application Publication
(10) Pub. No.: US 2026/0000867 A1
(43) Pub. Date: Jan. 1, 2026

(54) CATHETER TORQUING DEVICE AND RELATED METHODS

(71) Applicant: ASAHI INTECC CO., LTD., Seto-shi (JP)

(72) Inventors: Michael LUK, Orange, CA (US); Andy DENISON, Temecula, CA (US); Jeff BURKE, Fallbrook, CA (US); Kevin MAGRINI, Temecula, CA (US); Peter D'AQUANNI, Murrietta, CA (US); Robert RAGLAND, Brock, TX (US)

(73) Assignee: ASAHI INTECC CO., LTD., Seto-shi (JP)

(21) Appl. No.: 19/251,500

(22) Filed: Jun. 26, 2025

Related U.S. Application Data
(60) Provisional application No. 63/666,046, filed on Jun. 28, 2024.

(51) Int. Cl. A61M 25/01

(52) U.S. Cl. CPC A61M 25/01; A61M 2205/58

(57) ABSTRACT

A catheter torquing device is provided that enables a physician to rotate (e.g., torque) a catheter using a single hand. The provided catheter torquing device includes a catheter holder that can be slid onto a catheter. The catheter holder is adjustable between an engaged state, in which the catheter holder tightly grips the catheter, and a disengaged state, in which the catheter holder releases its grip on the catheter. When the catheter holder is in the engaged state, rotation of at least a portion of the catheter holder also rotates the catheter, whereas when the catheter holder is in the disengaged state, the catheter holder and the catheter can be translated relative to one another. The catheter torquing device further includes a base member that may be positioned directly onto a middle portion of a catheter and coupled to the catheter holder.

### CROSS-REFERENCE TO RELATED APPLICATIONS

This application claims the earlier filing date benefit of U.S. Provisional Application No. 63/666,046, filed on Jun. 28, 2024, the entirety of which is incorporated by reference herein.

### FIELD OF INVENTION

The present application relates generally to catheters. More specifically, the present application provides devices for rotating (i.e., applying torque to) a catheter and related methods.

### BACKGROUND

Catheters are thin, flexible tubes designed for medical purposes. Catheters come in various types and sizes tailored to specific medical needs and are used in a wide variety of medical procedures. Rotation of a catheter while a distal portion of the catheter is within the body is often needed in these medical procedures in order, for example, to help the catheter navigate to a desired location within the patient's body, such as within their vasculature.

### SUMMARY

The following summarizes some embodiments of the present disclosure to provide a basic understanding of the discussed technology. This summary is not an extensive overview of all contemplated features of the disclosure and is intended neither to identify key or critical elements of all embodiments of the disclosure nor to delineate the scope of any or all embodiments of the disclosure. Its sole purpose is to present some concepts of one or more embodiments of the disclosure in summary form as a prelude to the more detailed description that is presented later.

A new and innovative catheter torquing device is provided that enables a user (e.g., physician) to rotate (e.g., torque) a catheter using a single hand. The provided catheter torquing device includes a catheter holder that can be slid onto a catheter. The catheter holder is adjustable between an engaged state, in which the catheter holder grips the catheter (e.g., tightly enough that the catheter holder will not move (a) axially along the catheter when the catheter holder is advanced or retracted axially, and/or (b) rotationally about the catheter when the catheter holder is rotated), and a disengaged state, in which the catheter holder releases its grip on the catheter. When the catheter holder is in the engaged state, rotation of at least a portion of the catheter holder also rotates the catheter, whereas when the catheter holder is in the disengaged state, the catheter holder and the catheter can be translated relative to one another.

The catheter holder includes a gripping element (e.g., a multi-arm gripping element) that physically grips or releases the catheter, and is therefore also adjustable between the engaged and disengaged states. For example, the gripping element may be a collet. The catheter holder further includes a depressible trigger, which can be used to alter the gripping element's state. In an example, when the trigger is at rest, the gripping element engages the catheter, whereas when the trigger is depressed, the trigger forces the gripping element into the disengaged state such that the gripping element releases (or disengages) the catheter.

The catheter torquing device further includes a base member (e.g., a palm-sized structure) that can be positioned over the catheter and coupled to the catheter holder. With the base member coupled to the catheter holder, the base member can be positioned in the palm of a physician's hand while the physician uses two fingers of the same hand (e.g., thumb and index finger) to either rotate a portion of the catheter holder or depress the trigger and translate the catheter torquing device or the catheter. In some examples, the catheter torquing device can be positioned on the catheter adjacent to the introducer sheath through which the catheter enters the patient's body, which enables applying most or all of the torque to the distal portion of the catheter within the body.

In an example, a catheter torquing device includes a catheter holder and a base member. The catheter holder is configured to selectively grip a portion of a catheter when the portion of the catheter is disposed through the catheter holder, and includes a rotation element, a gripping element (e.g., a collet), and a release trigger. The rotation element includes a channel. The collet is disposed within the channel and rotationally coupled to the rotation element. The collet is transitionable between an engaged state and a disengaged state, and the collet is rotationally coupled to the catheter when in the engaged state and can translate axially relative to the catheter when in the disengaged state and the catheter is disposed through the catheter holder. The release trigger is disposed partially within the channel and transitionable between a triggered state and an untriggered state so as to transition the collet between the engaged state and the disengaged state. When the release trigger is in the triggered state, the collet is in the disengaged state, and when the release trigger is in the untriggered state, the collet is in the engaged state. The base member may be coupled with the catheter holder such that the rotation element is rotatable relative to the base member.

In another example, a catheter torquing device includes a rotation element, a release trigger, and a collet. The rotation element has a rotation axis. The release trigger is disposed partially within the rotation element and translatable relative to the rotation element along the rotation axis between a triggered state and an untriggered state. The release trigger is biased toward the untriggered state. The collet is disposed within the rotation element. When a catheter is disposed through the collet and the release trigger is in the triggered state, the collet is disengaged from the catheter, and when the release trigger is in the untriggered state, the collet engages the catheter so as to rotationally couple the catheter to the rotation element.

In another example, a catheter torquing device includes a catheter holder and a base member. The catheter holder is able to selectively grip a catheter when the catheter is disposed through the catheter holder such that, when the catheter holder is in an engaged state, rotation of at least a portion of the catheter holder rotates the catheter about an axis, and when the catheter holder is in a disengaged state, the catheter holder can translate relative to the catheter along the axis. The catheter holder includes a depressible trigger that, when depressed, transitions the catheter holder to the disengaged state. The base member may be coupled with the catheter holder such that at least a portion of the catheter holder is rotatable relative to the base member, which includes a channel that can receive the catheter when the catheter is disposed through the catheter holder. The catheter torquing device is configured and sized such that, when the catheter is disposed through the catheter holder and the base member is coupled to the catheter holder, a user can hold the catheter torquing device and rotate the catheter holder or depress the depressible trigger with only a single hand of the user.

In another example, a catheter adjustment method includes positioning a catheter through a catheter holder of a catheter torquing device. The catheter holder includes a collet that rotationally couples the catheter to the catheter holder when the collet is in an engaged state and that allows translation of the catheter holder relative to the catheter when the collet is in a disengaged state. The method further includes positioning a base member of the catheter torquing device around the catheter, coupling the base member to the catheter holder, and rotating the catheter by rotating, with only a single hand of a user, at least a portion of the catheter holder relative to the base member.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “end” refers to a concluding portion of the referenced structure and is not limited to the terminus of the referenced structure. Herein, translational movement, unless context indicates otherwise, is movement proximal or distal to the operator. The term “substantially” is defined as largely but not necessarily wholly what is specified—and include what is specified—as understood by a person of ordinary skill in the art. In any disclosed embodiment, the term “substantially” may be substituted with “within [a percentage] of” what is specified, where the percentage includes 0.1, 1, 5, and 10 percent.

Furthermore, all numerical ranges herein should be understood to include all integers, whole or fractions, within the range, inclusive of the ends of the ranges. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The terms “comprise” and any form thereof such as “comprises” and “comprising,” “have” and any form thereof such as “has” and “having,” and “include” and any form thereof such as “includes” and “including” are open-ended linking verbs. As a result, an apparatus or system that “comprises,” “has,” or “includes” one or more elements possesses those one or more elements but is not limited to possessing only those elements. Likewise, a method that “comprises,” “has,” or “includes” one or more steps possesses those one or more steps but is not limited to possessing only those one or more steps.

Any embodiment of any of the apparatuses, systems, and methods can consist of or consist essentially of—rather than comprise/have/include-any of the described steps, elements, and/or features. Thus, in any of the claims, the term “consisting of” or “consisting essentially of” can be substituted for any of the open-ended linking verbs recited above in order to change the scope of a given claim from what it would otherwise be using the open-ended linking verb.

An apparatus or system that is configured in a certain way is configured in at least that way, but it can also be configured in other ways than those specifically described. Furthermore, reference in the appended claims to an apparatus or system or a component of an apparatus or system being adapted to or configured to perform a particular function encompasses that apparatus, system, or component, whether or not it or that particular function is activated, turned on, or unlocked, as long as that apparatus, system, or component is so adapted or configured.

The feature or features of one embodiment may be applied to other embodiments, even though not described or illustrated, unless expressly prohibited by this disclosure or the nature of the embodiments.

Some details associated with the embodiments are described above and others are described below.

Additional features and advantages of the disclosed catheter torquers and related methods are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

The following drawings illustrate by way of example and not limitation. For the sake of brevity and clarity, every feature of a given structure is not always labeled in every figure in which that structure appears. Similar components or features may have the same reference label. Further, various components of the same type may be distinguished by following the reference label with a second label that distinguishes among the similar components. Identical reference numbers do not necessarily indicate an identical structure. Rather, the same reference number may be used to indicate a similar feature or a feature with similar functionality, as may non-identical reference numbers.

FIG. 1 is a box diagram of a catheter torquing device, according to an aspect of the present disclosure.

FIG. 2 is a perspective view of the catheter torquing device, according to an aspect of the present disclosure.

FIG. 3 is a perspective view of a catheter holder of the catheter torquing device, according to an aspect of the present disclosure.

FIG. 4 is a perspective view of a base member of the catheter torquing device, according to an aspect of the present disclosure.

FIG. 5A is a side view of the catheter holder with the rotation element shown in cross-section, according to an aspect of the present disclosure.

FIG. 5B is a magnified cross-section of the catheter holder with a catheter disposed therethrough, according to an aspect of the present disclosure.

FIG. 6 is a cross-section of the base member, according to an aspect of the present disclosure.

FIG. 7 is a perspective view of the catheter holder disposed on a catheter and the base member in the process of being disposed on the catheter, according to an aspect of the present disclosure.

FIG. 8A is a schematic showing another embodiment of the catheter holder being disposed adjacent a strain relief of a catheter, according to an aspect of the present disclosure.

FIG. 8B is a schematic showing the catheter holder of FIG. 8A being disposed adjacent an introducer sheath through which a catheter is disposed, according to an aspect of the present disclosure.

FIG. 9 is a perspective view of a user holding one of the present catheter torquing devices and depressing the trigger, according to an aspect of the present disclosure.

FIG. 10 is a perspective view of a user holding one of the present catheter torquing devices and rotating the rotation element, according to an aspect of the present disclosure.

FIG. 11 is a perspective view of an alternative base member, according to an aspect of the present disclosure.

### DETAILED DESCRIPTION

The detailed description set forth below, in connection with the appended drawings, is intended as a description of various configurations and is not intended to limit the scope of the disclosure. Rather, the detailed description includes specific details for the purpose of providing a thorough understanding of the inventive subject matter. It will be apparent to those skilled in the art that these specific details are not required in every case and that, in some instances, well-known structures and components are shown in block diagram form for clarity of presentation.

A catheter torquing device is provided that enables a physician to rotate (e.g., torque) a catheter using a single hand. Conventionally, rotation of a catheter is mainly accomplished through rotating a hub coupled to the catheter. Much of the torque applied to the catheter by this method, however, results in twisting the proximal portion of the catheter that is outside of the introducer sheath and therefore outside of the patient's body, rather than twisting the distal portion of the catheter inside the patient's body. Other conventional devices for rotating a catheter undesirably require the physician to use two hands.

The provided catheter torquing device includes a catheter holder that can be slid onto a catheter. The catheter holder is adjustable between an engaged state, in which the catheter holder tightly grips the catheter, and a disengaged state, in which the catheter holder releases its grip on the catheter. When the catheter holder is in the engaged state, rotation of at least a portion of the catheter holder also rotates the catheter, whereas when the catheter holder is in the disengaged state, the catheter holder and the catheter can be translated relative to one another.

The catheter holder includes a rotation element, a trigger, and a gripping element. The gripping element is a clamping device, such as a collet, that can be used to securely hold a component within the clamping device in place. For instance, the gripping element is disposed within the rotation element and is the portion of the catheter holder that physically grips or releases the catheter. As such, the gripping element is also adjustable between the engaged and disengaged states. The gripping element includes a plurality of arms defining a cylindrical inner surface that matches the catheter's shape, thereby ensuring the arms have a tight grip on the catheter when the gripping element is tightened.

The trigger is used to alter the state the gripping element is in. For example, the trigger may be biased (e.g., via a spring) toward an untriggered state and the gripping element may be biased (e.g., via a spring) toward the engaged state such that, when the trigger is at rest, the gripping element engages the catheter. Conversely, when the trigger is depressed into the triggered state, the trigger forces the gripping element into the disengaged state such that the gripping element releases the catheter. The rotation element is rotationally coupled to the gripping element so that rotating the rotation element with the gripping element in the engaged state rotates the catheter.

The catheter torquing device further includes a base member that can be positioned over the catheter and coupled to the catheter holder. For instance, the base member can be quickly placed on the catheter and coupled to the catheter holder, or quickly removed from both, while the catheter holders remains on the catheter, so that the base member is only on the catheter when needed. With the base member coupled to the catheter holder, and more particularly to the rotation element, the base member can be positioned in the palm of a physician's hand while the physician can use two fingers of the same hand (e.g., thumb and index finger) to either rotate the rotation element or depress the trigger and translate the catheter torquing device relative to the catheter. In some examples, the catheter torquing device can be positioned on the catheter adjacent to the introducer sheath through which the catheter enters the patient's body, which enables applying most or all of the torque to the distal portion of the catheter within the body.

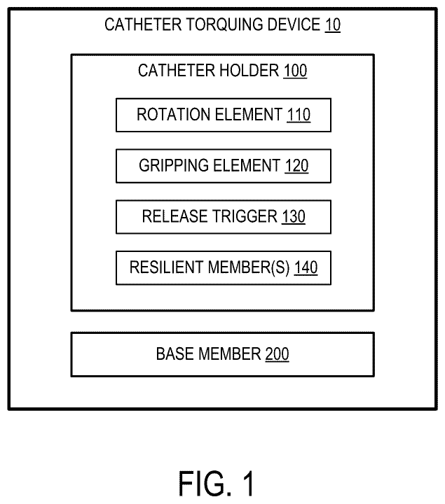

FIG. 1 is a box diagram of a catheter torquing device 10. The catheter torquing device 10 includes a catheter holder 100 that can be used to grip or release a catheter when the catheter is disposed through the catheter holder 100. The catheter holder 100 can also be used to rotate a gripped catheter. For instance, the catheter holder 100 includes a gripping element 120 disposed within a rotation element 110. The gripping element 120 is a clamping device that can be adjusted between an engaged state in which the clamping device can securely grip a catheter disposed through the clamping device, and a disengaged state in which the clamping device and the catheter can translate relative to one another. For example, the gripping element 120 may be a collet. The gripping element 120 is rotationally coupled to the rotation element 110 such that when the gripping element 120 securely grips the catheter, rotating the rotation element 110 rotates the catheter.

A release trigger 130 (also referred to herein as a trigger) of the catheter holder 100 can be used to change the state of the gripping element 120. For instance, the release trigger 130 can be transitioned between an untriggered state and a triggered state to transition the gripping element 120 between the engaged state and the disengaged state. In an example, the release trigger 130 is biased toward the untriggered state and the gripping element 120 is biased toward the engaged state. For instance, the catheter holder 100 includes one or more resilient members 140. In an example, a first resilient member of the one or more resilient members 140 biases the release trigger 130 toward the untriggered state and a second resilient member of the one or more resilient members 140 biases the gripping element 120 toward the engaged state. In this example, depressing the release trigger 130 transitions the release trigger 130 to the triggered state, which transitions the gripping element 120 to the disengaged state. The release trigger 130 may be partially disposed within the rotation element 110 in some embodiments.

The catheter torquing device 10 further includes a base member 200. The base member 200 can be coupled to the catheter holder 100, and more particularly to the rotation element 110, such that the rotation element 110 can rotate relative to the base member 200 when coupled. The base member 200 can be positioned directly onto a middle portion of a catheter, as opposed to being slid onto a catheter from the catheter's distal or proximal ends. As such, the base member 200 may be positioned on a catheter subsequent to the catheter holder 100 already being positioned on the catheter.

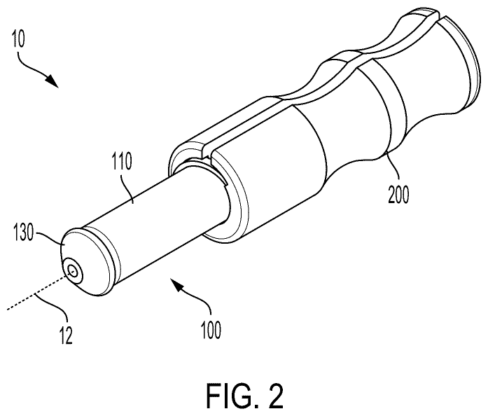

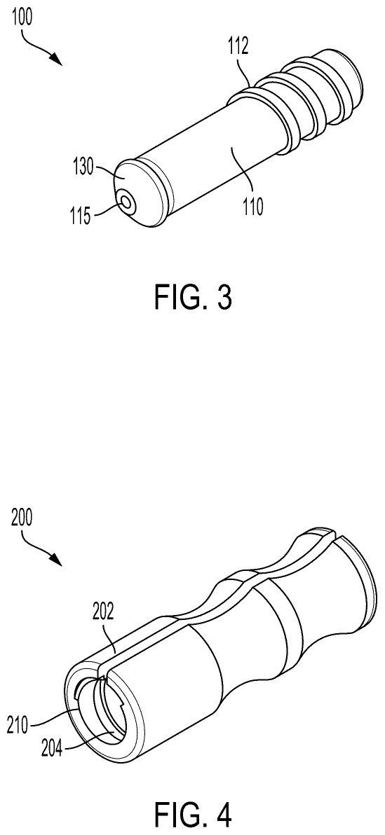

FIGS. 2 to 6 show an example implementation of the catheter torquing device 10. In this example, the rotation element 110 is an elongate, cylindrical structure defining a channel 114 extending therethrough. When the catheter holder 100 is assembled, the rotation element 110 is able to rotate about a rotation axis 12. The channel 114 includes a widened portion 116 and a widened portion 118. The widened portion 118 includes an angled surface 119 that is disposed at an angle relative to the rotation axis 12. In an example, the angled surface 119 may be cone-shaped. The angle may be a suitable angle for the depicted use that is within any of the following ranges: greater than 0° and less than 90°, 25° to 65°, 35° to 55°, or 40° to 50°. In an example, the angle may be 45° or about 45°. At least one male thread 112 extends from a proximal portion of an exterior surface of the rotation element 110. In other examples, the proximal portion of the rotation element 110 may alternatively include a protrusion or at least one female thread. The proximal portion of the rotation element 110 includes a proximal end 113 and a portion of the rotation element 110 that extends distally from the proximal end 113.

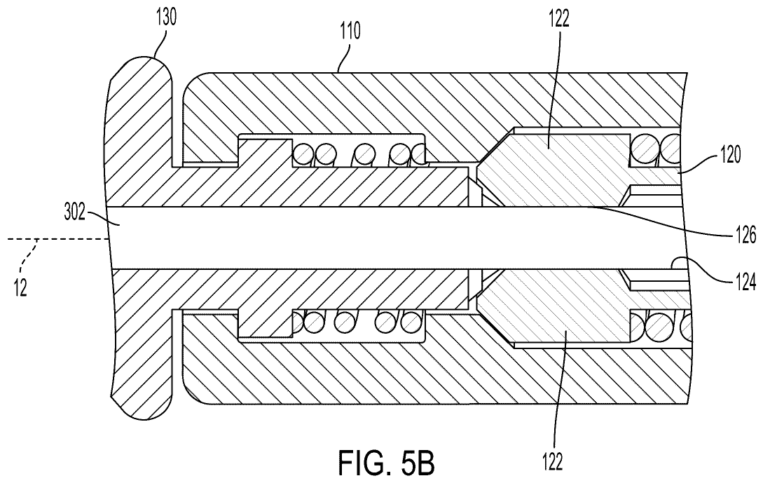

As shown in FIGS. 5A and 5B, the gripping element 120 is an elongate structure that defines a channel 124 (e.g., a passageway extending through the gripping element 120) therethrough and includes a plurality of arms 122. When the catheter holder 100 is assembled, the gripping element 120 is disposed within the channel 114 of the rotation element 110 and is able to rotate about the rotation axis 12. For instance, at least a portion of the plurality of arms 122 is disposed within the widened portion 118 of the channel 114. A resilient member 140B biases the gripping element 120 toward a distal end 111 of the rotation element 110 (e.g., toward a distal end of the widened portion 118 of the channel 114). For instance, the resilient member 140B is disposed between a surface of the gripping element 120 (e.g., a proximally facing surface comprising portions of each arm 122) and a surface of the rotation element 110 (e.g., a distally-facing surface of the rotation element 110, such as a shoulder formed by the transition of the widened portion 118 to the channel 114).

Each arm of the plurality of arms 122 can be flexed toward the rotation axis 12 from a resting position and allowed to return to the resting position when no longer flexed, such as when the arms are driven proximally by the proximal end of the release trigger 130. One possible resting position of the arms 122 is that they are biased away from the rotation axis 12 such that interior surfaces 126 of the arms 122 are non-parallel to the rotation axis 12. From this resting position, when the arms 122 are radially compressed, the interior surfaces 126 become parallel to the rotation axis 12 such that at least a lengthwise portion of each of the interior surfaces 126 is in contact with a lengthwise portion (having the same length, or substantially the same length, as the lengthwise portion of the respective interior surface 126, which length may comprise a value from 1 to 15 millimeters) of a catheter 302 disposed through the channel 124 of the gripping element 120. FIG. 5B depicts the gripping element 120 in an engaged state with the interior surfaces 126 lined up against the catheter 302 so as to grip the catheter 302. In another possible resting position, the interior surfaces 126 of the arms 122 may be parallel to the rotation axis 12, and when the arms 122 are radially compressed, the interior surfaces 126 remain parallel to the rotation axis 12.

In an example, each arm of the plurality of arms 122 is machined out of a body of the gripping element 120 such that a slit separates adjacent arms of the plurality of arms 122. For example, the gripping element 120 may comprise metal such that the arms 122 have resiliency or are otherwise capable of elastically returning to their resting (non-flexed) positions when not forced distally against the angle surface 119. The slits enable the arms 122 to flex toward the rotation axis 12.

In at least some embodiments, such as the illustrated embodiment, each arm of the plurality of arms 122 includes an angled surface 123 that is at an angle to the rotation axis 12 when the gripping element 120 is disposed within the rotation element 110 as shown. In an example, the angled surface 123 may be cone-shaped. The angle may be a suitable angle for the depicted use that is within any of the following ranges: greater than 0° and less than 90°, 25° to 65°, 35° to 55°, or 40° to 50°. In an example, the angle may be 45° or about 45°. The angle of the angled surface 123 may be equal to the angle of the angled surface 119.

With continued reference to FIG. 5A, the release trigger 130, in this example, includes a cap 132 and a stem 134 extending from the cap 132. Each of the cap 132 and the stem 134 define a channel 115 extending therethrough. A protrusion 136 extends from the stem 134. When the catheter holder 100 is assembled, at least a portion of the stem 134 is disposed within the channel 114 of the rotation element 110, whereas the cap 132 is disposed outside of the rotation element 110. A resilient member 140A biases the release trigger 130 toward the distal end 111 of the rotation element 110 (e.g., toward a distal end of the widened portion 116 of the channel 114). For instance, the resilient member 140A is disposed between a surface of the protrusion 136 and a surface of the rotation element 110 (e.g., a proximally-facing shoulder at the transition of the widened portion 116 to the more narrow size of channel 114). The protrusion 136 limits translational movement of the release trigger 130 along the rotation axis 12 by contacting the proximal end and distal end (e.g., the shoulders adjacent those ends) of the widened portion 116.

In an example, the resilient members 140A, 140B may be any suitable mechanical device capable of elastically storing mechanical energy when compressed and thereafter releasing that stored mechanical energy, such as a spring. In various embodiments, each of the resilient members 140A, 140B may be a coil spring or a machined spring, though other types of springs are possible.

Operation of the catheter holder 100 will now be described. With the release trigger 130 at rest, the resilient member 140A biases the release trigger 130 into the untriggered state and the resilient member 140B biases the gripping element 120 into the engaged state. In more detail, a diameter of the widened portion 118 narrows along the angled surface 119 toward the distal end of the widened portion 118. The resilient member 140B forces the angled surface 123 of the plurality of arms 122 into this narrowing segment of the widened portion 118 such that a surface of the rotation element 110 forces the plurality of arms 122 to flex toward the rotation axis 12. When a catheter is disposed through the gripping element 120, the plurality of arms 122 in this flexed, or engaged, state tightly grip the catheter so as to rotationally couple the catheter to the gripping element 120 and limit (e.g., prevent) translation of the catheter or gripping element 120 relative to one another. In some embodiments, the release trigger 130 contacts the gripping element 121 when in the untriggered state as depicted. In other embodiments, a gap may exist between the release trigger 130 and the gripping element 120 when the release trigger 130 is in the untriggered state.

The gripping element 120 is rotationally coupled to the rotation element 110. For example, the gripping element 120 may include a non-cylindrical portion that conforms to a non-cylindrical portion of the channel 114 such that rotation of the rotation element 110 also rotates the gripping element 120. Other techniques for rotationally coupling the rotation element 110 and the gripping element 120 are possible. For example, the friction between the gripping element 120 and the rotation element 110 may be sufficiently high when the trigger 130 is in its resting state and the gripping element 120 is engaged with a catheter so that rotation of the rotation element 110 translates to rotation of the gripping element 120 (and, thus, the catheter too) without further interference between a portion of the gripping element 120 and the rotation element 110.

When the cap 132 of the release trigger 130 is depressed toward the rotation element 110 along the rotation axis 12, the release trigger 130 enters the triggered state. In the triggered state, the release trigger 130 compresses the resilient member 140A and forces the gripping element 120 to compress the resilient member 140B by pushing the gripping element 120 toward the proximal end 113 of the rotation element 110. The plurality of arms 122 are thereby released from the flexed, engaged state and transition to the disengaged state as the arms 122 expand into the widened portion 118. When a catheter is disposed through the gripping element 120, the plurality of arms 122 in the disengaged state allow the catheter torquing device 10 (and more specifically the catheter holder 100) to translate along the rotation axis 12, or rotate about the rotation axis 12, relative to the catheter.

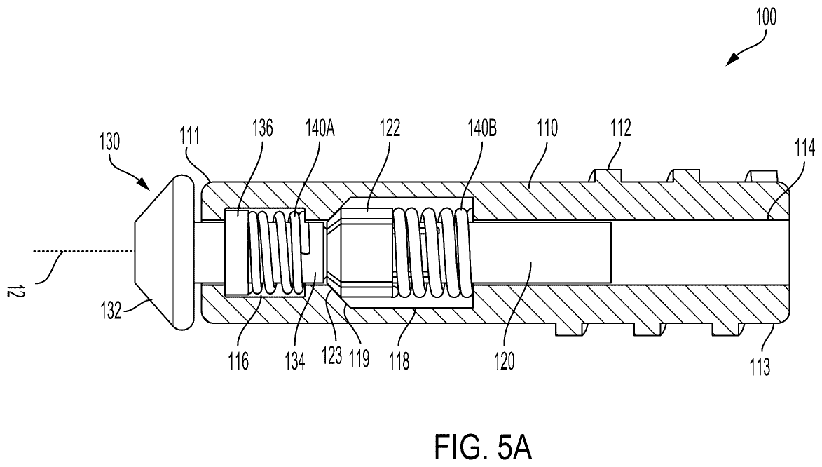

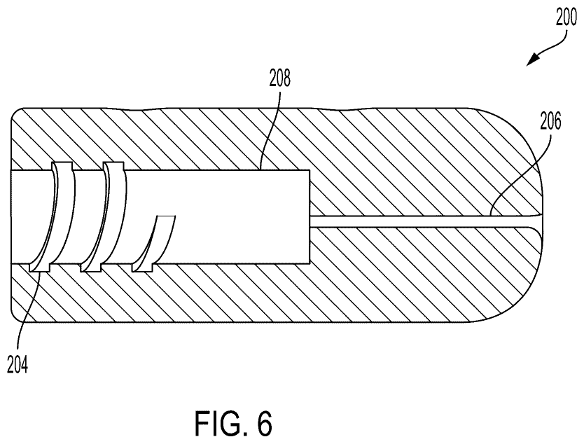

With reference to FIGS. 4 and 6, the base member 200, in the depicted example, is an elongate structure defining a channel including a first channel portion 206 and a second channel portion 208. The first channel portion 206 is sized to allow passage therethrough of a catheter. The second channel portion 208 is sized to receive a proximal portion of the rotation element 110 and, in this example, includes at least one female thread 204. In other examples, the second channel portion 208 may alternatively include at least one male thread. The depicted embodiment of the base member 200 further includes a slit 202 extending from an exterior surface of the base member 200 to an interior surface of the base member 200 defining the channel. As will be described further below, the slit 202 enables passing a middle portion of catheter through the slit 202 and into the channel. Other suitable structures for positioning the base member 200 directly onto a middle portion of a catheter are possible. In various aspects, the exterior of the base member 200 may be shaped to ergonomically fit a user's hand.

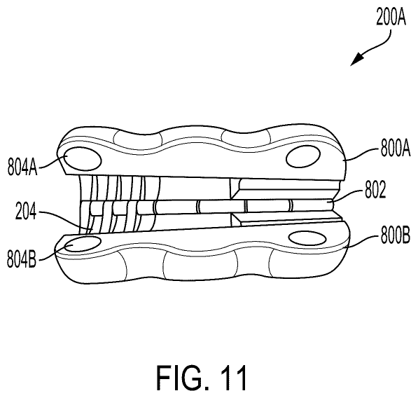

For example, FIG. 11 depicts an alternative embodiment of the base member, shown as base member 200A. In this alternative embodiment, the base member 200A includes a first body portion 800A and a second body portion 800B that are joined at a hinge 802 so as to be pivotable relative to one another. With this pivoting action, the first body portion 800A and the second body portion 800B can be separated from one another to position the base member 200A over a catheter, including over one of the present catheter holders that has been positioned over a catheter, and then pivoted closed so that the catheter (and catheter holder, if present) is enclosed within a channel of the base member 200A similar to the channel of the base member 200. In various aspects, the first body portion 800A and the second body portion 800B may be maintained closed by at least one set of magnets 804A, 804B. In other aspects, the at least one set of magnets 804A, 804B may be replaced by a clasp, latch, or other suitable mechanism.

Returning to FIG. 2, the base member 200 may be coupled to the catheter holder 100 such that at least a portion of the catheter holder 100 can rotate about the rotation axis 12 relative to the base member 200 when coupled. For example, the base member 200 may be coupled to the rotation element 110 by engaging the at least one female thread 204 of the base member 200 with the at least one male thread 112 of the rotation element 110. When the rotation element 110 is rotated about the rotation axis 12 relative to the base member 200, the at least one male thread 112 advances or retracts along the at least one female thread 204. As described above, the at least one male thread 112 may alternatively be a protrusion capable of traveling through and along the at least one female thread 204.

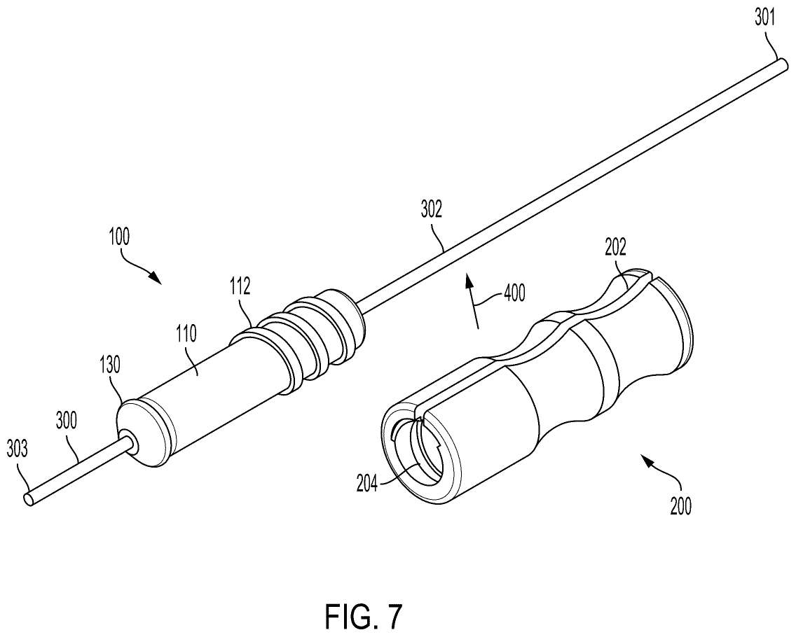

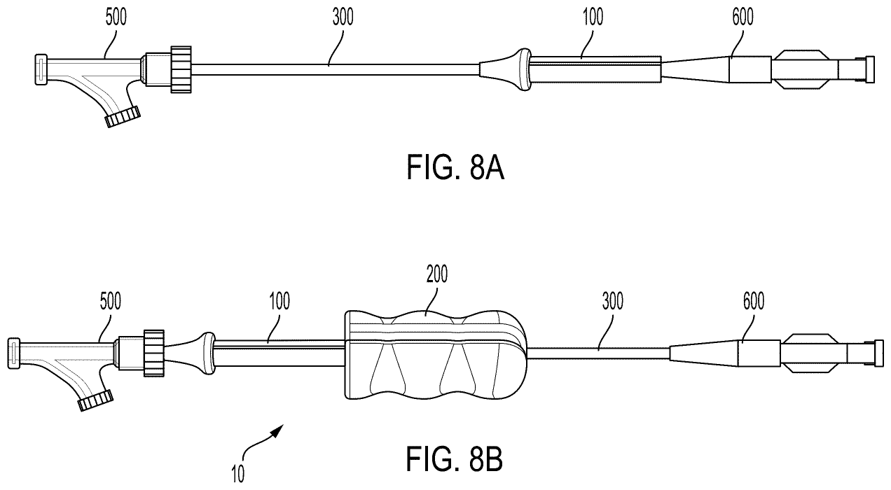

A usage case of the catheter torquing device will now be described. Referring to FIG. 7, a physician (or other individual) may slide the catheter holder 100 onto a catheter 300 while depressing the release trigger 130. For example, the physician may advance the catheter holder 100 over a distal end 301 or a proximal end 303 of the catheter 300 so that the catheter 300 extends through each of the channels of the rotation element 110, the gripping element, and the release trigger 130 as depicted. In some embodiments, the catheter holder 100 may be preloaded onto the catheter 300 in a medical kit provided to a physician. For example, as shown in FIG. 8A, the catheter holder 100 (shown with an alternative embodiment of the trigger element in which the cap has an axially-longer shape than the cap 132 shown in FIGS. 5A and 5B) may be preloaded onto the catheter 300 such that it is positioned adjacent a strain relief 600 of the catheter 300. In such embodiments, the physician does not need to slide the catheter holder 100 onto the catheter 300 since it is already positioned on the catheter 300. The catheter 300 may be any suitable type of catheter, such as a single operator exchange style catheter or an over-the-wire type catheter.

Returning to FIG. 7, a distal portion of the catheter 300 is thereafter inserted into a patient's body, such as through an introducer sheath. At this stage, the catheter holder 100 is positioned on a middle portion 302 of the catheter 300 that remains outside the patient's body. It will be appreciated that the catheter 300 is not shown to scale in any of the figures in which it is depicted.

When the physician desires to rotate the catheter 300 with the catheter torquing device, the physician may position the base member 200 directly onto the middle portion 302 of the catheter 300. For example, the physician may bring the base member 200 toward the catheter 300 in the direction of arrow 400 such that the middle portion 302 passes through the slit 202 and into the channel of the base member 200. In another example, the physician may bring the base member 200A toward the catheter 300 and then close the first and second body portions 800A, 800B around the middle portion 302. Positioning the base member 200 “directly” onto the middle portion 302 as that term is used herein means that the base member 200 does not have to be advanced over the distal end 301 or the proximal end 303 to be positioned on the catheter 300.

In some embodiments, the physician may reposition the catheter holder 100 along the catheter 300 prior to positioning the base member 200 directly onto the middle portion. For example, from the preloaded configuration depicted in FIG. 8A in which the catheter holder 100 is positioned adjacent the strain relief 600, the physician may reposition the catheter holder 100 toward the distal end of the catheter 300 to make room for the base member 200. To do so, the physician depresses the release trigger 130, translates the catheter holder 100 along the catheter 300 to a desired position, and then releases the release trigger 130. The physician can then couple the base member 200 to the rotation element 110 via the at least one male thread 112 of the rotation element and the at least one female thread 204 of the base member 200. The base member 200 need only be positioned on the catheter 300 and coupled to the rotation element 110 to fully assemble the catheter torquing device when the physician desires to rotate the catheter 300.

In some embodiments, with the assembled catheter torquing device, the physician can then rotate the catheter 300 by rotating the rotation element 110 relative to the base member 200, while the release trigger 130 is at rest in the untriggered state. In an example, the physician can rotate the catheter 180° in either direction so that a full 360° of a vessel in which the catheter is positioned can be reached by the catheter. For example, the physician may desire to aspirate on all sides of the vessel.

In other embodiments, the physician may reposition the catheter torquing device along the catheter 300 prior to rotating the catheter 300. For example, the physician may reposition the catheter torquing device so that the catheter holder 100 (i.e., the cap 132 of the release trigger 130) is positioned adjacent to an introducer sheath 500 used to introduce the catheter 300 into the patient's body, as depicted in FIG. 8B. Positioning the catheter holder 100 adjacent to the introducer sheath 500 reduces the likelihood of losing torque that is otherwise applied to a proximal end of the catheter (through, e.g., lost motion of the catheter) by applying torque to the portion of the catheter 300 close to the portion of the catheter that is within the body. Focusing the torque in this manner enables the physician to have greater rotational control of the portion of the catheter 300 within the body.

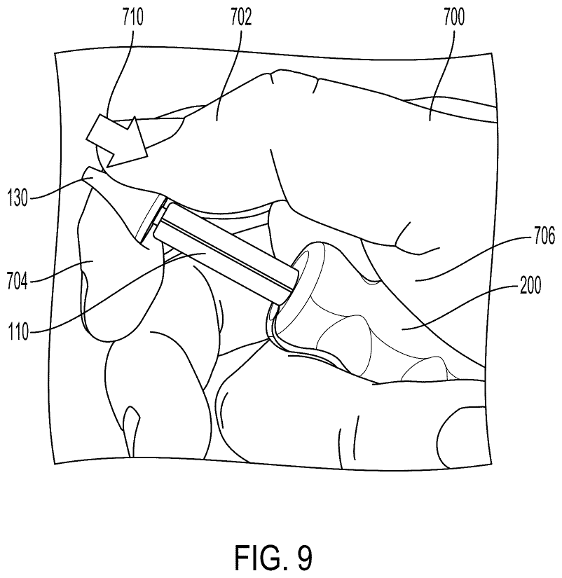

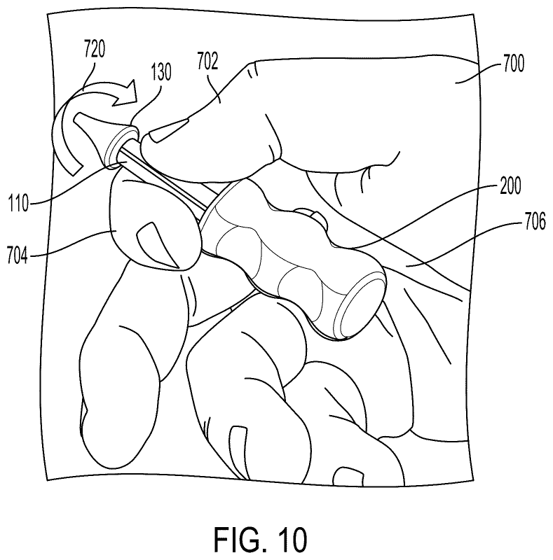

With the catheter torquing device in a position for operation, the physician can operate the catheter torquing device 10 using a single hand. For example, FIG. 9 depicts the catheter torquing device held in a hand 700 of a user. While the user holds the base member 200 in a palm 706 of their hand 700, the user can use their thumb 702 and index finger 704 to depress the release trigger 130 in the direction of arrow 710. It will be appreciated that the user may use a different set of two fingers, such as their thumb and middle finger, or may use three fingers, such as their thumb, index finger, and middle finger. In another example, FIG. 10 depicts the user rotating the rotation element 110 using their thumb 702 and index finger 704. The user's middle finger, ring finger, and pinky finger are depicted open to show the base member 200 in the palm 706, but at least one of the middle finger, ring finger, and pinky finger may be closed around the base member 200 during operation, such as to maintain the base member 200 stationary relative to the rotation element 110.

In some embodiments, the physician may advance the catheter further into the patient's body by depressing the release trigger with one of the physician's hands and then advancing the catheter relative to the catheter holder with the physician's other hand. In some embodiments, after rotating the catheter is complete, the physician may decouple the base member from the catheter holder and remove the base member from the catheter, which can be done with a single hand. In some embodiments, after rotating the catheter is complete, the physician may reposition the catheter holder away from the introducer sheath until catheter rotation is needed again.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A catheter torquing device comprising:
a catheter holder configured to selectively grip a portion of a catheter when the portion of the catheter is disposed through the catheter holder, the catheter holder including:
a rotation element including a channel;
a collet disposed within the channel and rotationally coupled to the rotation element, wherein the collet is transitionable between an engaged state and a disengaged state, and the collet is rotationally coupled to the catheter when in the engaged state and can translate axially relative to the catheter when in the disengaged state and the catheter is disposed through the catheter holder; and
a release trigger disposed partially within the channel and transitionable between a triggered state and an untriggered state so as to transition the collet between the engaged state and the disengaged state, wherein when the release trigger is in the triggered state, the collet is in the disengaged state, and when the release trigger is in the untriggered state, the collet is in the engaged state; and
a base member configured to be coupled with the catheter holder such that the rotation element is rotatable relative to the base member.

2. The catheter torquing device of claim 1, wherein:
the collet includes a plurality of arms;
the channel of the rotation element includes a first portion having a first diameter and a second portion having a second diameter greater than the first diameter;
when the plurality of arms are disposed in the first portion, the plurality of arms are compressed toward a rotation axis of the rotation element such that the collet is in the engaged state; and
when the plurality of arms are disposed in the second portion, the plurality of arms expand away from the rotation axis such that the collet is in the disengaged state.

3. The catheter torquing device of claim 2, wherein the first portion is cone-shaped.

4. The catheter torquing device of claim 1, wherein translating the release trigger toward the rotation element along a rotation axis of the rotation element transitions the release trigger to the triggered state and the collet to the disengaged state.

5. The catheter torquing device of claim 1, wherein the rotation element includes a protrusion and the base member includes a female thread, and the protrusion is configured to be disposed within the female thread to thereby couple the base member and the catheter holder.

6. The catheter torquing device of claim 1, wherein the collet is biased toward the engaged state and the release trigger is biased toward the untriggered state.

7. The catheter torquing device of claim 6, wherein the catheter holder further comprises:
a first resilient member disposed within the channel of the rotation element and configured to bias the release trigger toward the untriggered state; and
a second resilient member disposed within the channel and configured to bias the collet toward the engaged state.

8. The catheter torquing device of claim 7, wherein each of the first resilient member and the second resilient member is a spring.

9. The catheter torquing device of claim 1, configured such that a user may transition the collet between the engaged state and the disengaged state, and/or rotate the rotation element, with a single hand.

10. The catheter torquing device of claim 9, sized such that, with the base member positioned in a palm of the single hand, the user may transition the collet between the engaged state and the disengaged state, and/or rotate the rotation element, with only two fingers of the single hand.

11. The catheter torquing device of claim 1, wherein the base member includes a slit leading to a channel of the base member such that a portion of a catheter may be received through the slit and into the channel.

12. The catheter torquing device of claim 11, wherein the catheter holder is coupled to the base member within the channel.

13. A catheter torquing device comprising:
a rotation element having a rotation axis;
a release trigger disposed partially within the rotation element and translatable relative to the rotation element along the rotation axis between a triggered state and an untriggered state, wherein the release trigger is biased toward the untriggered state; and
a collet disposed within the rotation element;
wherein when a catheter is disposed through the collet and the release trigger is in the triggered state, the collet is disengaged from the catheter, and when the release trigger is in the untriggered state, the collet engages the catheter so as to rotationally couple the catheter to the rotation element.

14. The catheter torquing device of claim 13, further comprising a base member configured to be coupled to the rotation element such that the rotation element may rotate about the rotation axis relative to the base member.

15. The catheter torquing device of claim 14, wherein the base member includes a slit configured to receive a lengthwise portion of a catheter.

16. The catheter torquing device of claim 14, wherein the base member includes a first portion and a second portion that are pivotable relative to one another.

17. The catheter torquing device of claim 16, wherein the first portion and the second portion together form a channel when joined, and a portion of the rotation element is disposed within the channel.

18. A catheter torquing device comprising:
a catheter holder configured to selectively grip a catheter when the catheter is disposed through the catheter holder such that, when the catheter holder is in an engaged state, rotation of at least a portion of the catheter holder rotates the catheter about an axis, and when the catheter holder is in a disengaged state, the catheter holder can translate relative to the catheter along the axis, the catheter holder includes a depressible trigger that, when depressed, transitions the catheter holder to the disengaged state; and
a base member configured to be coupled with the catheter holder such that at least a portion of the catheter holder is rotatable relative to the base member, which includes a channel configured to receive the catheter when the catheter is disposed through the catheter holder;
wherein, the catheter torquing device is configured and sized such that, when the catheter is disposed through the catheter holder and the base member is coupled to the catheter holder, a user can hold the catheter torquing device and rotate the catheter holder or depress the depressible trigger with only a single hand of the user.

19. The catheter torquing device of claim 18, wherein the catheter torquing device is configured and sized such that, when the base member is disposed in a palm of the single hand of the user, the user can rotate the catheter holder and/or depress the depressible trigger with only two fingers of the single hand.

20. The catheter torquing device of claim 18, wherein the catheter holder includes a rotation element and a collet disposed within the rotation element.

21. A catheter adjustment method comprising:
positioning a catheter through a catheter holder of a catheter torquing device, wherein the catheter holder includes a collet that rotationally couples the catheter to the catheter holder when the collet is in an engaged state and that allows translation of the catheter holder relative to the catheter when the collet is in a disengaged state;
positioning a base member of the catheter torquing device around the catheter;
coupling the base member to the catheter holder;
rotating the catheter by rotating, with only a single hand of a user, at least a portion of the catheter holder relative to the base member.

22. The catheter adjustment method of claim 21, further comprising:
depressing a trigger of the catheter holder, thereby transitioning the collet from the engaged state to a disengaged state; and
translating the catheter holder along the catheter to a desired position.

23. The catheter adjustment method of claim 22, wherein the desired position is adjacent an introducer sheath through which the catheter is disposed.
